(19) United States
(12) Patent Application Publication
(10) Pub. No.: US 2021/0236582 A1
(43) Pub. Date: Aug. 5, 2021

(54) ANTIMICROBIAL COMPOSITION BASED ON POLYPHENOLS AND POLYSACCHARIDES, METHOD FOR PREPARING THEREOF AND USE OF THE SAME

(71) Applicant: NEARMEDIC INTERNATIONAL LIMITED, Nicosia (CY)

(72) Inventors: Vladimir Georgievich NESTERENKO, Moscow (RU); Anatoly Petrovich SUSLOV, Moscow (RU); Sergey Alexandrovich TSYRULNIKOV, Moscow (RU); Mikhail Nikolaevich NENASHEV, Moscow (RU); Irina Vladislavovna KISELEVA, Moscow reg. (RU); Vitaly Vladimirovich KUZNETSOV, Moscow (RU); Maria Veniaminovna KONOPLEVA, Moscow (RU)

(73) Assignee: NEARMEDIC INTERNATIONAL LIMITED, Nicosia (CY)

(21) Appl. No.: 17/056,602

(22) PCT Filed: May 17, 2019
(86) PCT No.: PCT/RU2019/050063

§ 371 Date: Nov. 18, 2020

(30) Foreign Application Priority Data
May 18, 2018 (RU) 2018118348

(51) Int. Cl. A61K 36/9068; A61K 31/721; A61K 31/717; A61K 31/05; A61K 36/28; A61K 36/484; A61K 31/215; A61P 31/04; A61P 31/16; C08B 11/20; C08B 37/02

(52) U.S. Cl. CPC A61K 36/9068; A61K 31/721; A61K 31/717; A61K 31/05; A61K 36/28; C08B 37/0021; A61K 31/215; A61P 31/04; A61P 31/16; C08B 11/20; A61K 36/484

(57) ABSTRACT

The present invention relates to an antimicrobial composition comprising a product of co-treatment of polyphenol and polysaccharide, and to a method for preparing this composition and an antiviral agent.

### FIELD OF THE INVENTION

The present invention relates to the field of organic chemistry and pharmaceuticals and is directed to antiviral and antimicrobial compositions comprising a polyphenol/polysaccharide co-treatment product, a method of preparing this composition and an antiviral agent.

### BACKGROUND

Natural polyphenols are objects of increased interest in the pharmaceutical industry because of a wide range of their pharmacological activity and structural diversity (J. Agric. Food Chem., 58, 10016-10019, (2010)).

In particular, they possess antitumor (Cancer Res., 49, 3754-3758, (1989)), antiviral (Contraception, 39, 579-587 (1989)), antiparasitic (Science 218, 288-289 (1982)), and anti-oxidant activities (J. Biol. Chem., 156, 633-642 (1944)).

However, disadvantages of polyphenols, which prevent their widespread use as medicaments, are their toxicity for the reproductive system, heart, and liver (Yin Juanjuan, A Dissertation for the Degree of Doctor of Philosophy The Graduate School of Clemson University, (2010)) and their poor solubility in water.

There are various approaches for overcoming these disadvantages.

The most common approach is a structural modification of polyphenols both without (Chem. Nat. Comp., 33, 545-547, (1998)) and with (J. Pharm. Sci., 64, 6, 1073-1075 (1975)) changes in substituents in the aromatic ring, or with changes in the aromatic core (Chem. Nat. Comp., 30, 1, 42-48, (1994)). However, the resulting new low-molecular weight compounds, despite a decreased toxicity, have a poor solubility in water and in each case require the development of methods for preparing and isolating a reaction product.

Another approach to improving solubility and reducing the toxicity of polyphenols is the preparation of non-covalent compositions with oligomeric and polymeric carriers: polyvinylpyrrolidone (Chem. Nat. Comp., 32, 2, 177-179 (1996)), glycyrrhizinic acid (Chem. Nat. Comp., 32, 2, 177-179 (1996)), cyclodextrins (Spectrochimica Acta, Part A, 61, 1025-1028, (2005)), as well as their encapsulation into polymeric micelles (U.S. Pat. No. 8,945,627). In this case, the starting compounds are present in the product in a structurally unchanged form. The possibilities of this approach are limited by the structural diversity of polyphenols and their derivatives.

The third approach allowing for overcoming the drawbacks of natural polyphenols is a change in the structure of polyphenols by covalent binding to a polymer carrier, an example of which is antiviral drug Kagocel®, in which the necessary physicochemical and biological properties are achieved by a simultaneous reduction in the number of aldehyde groups in the polysaccharide and polyphenol (patent RU 22700708). However, the limited structural diversity of functional groups of polyphenol and a high-molecular weight carrier, which are capable of covalently binding to each other, significantly limits the application of this approach. In addition, in some cases the resulting compounds demonstrate a reduced solubility.

Considering the above, there is still the problem of developing a controlled method for preparing compositions of polyphenols and polysaccharides, having improved physicochemical and biological properties compared with the starting components in order to obtain products with desired characteristics depending on the process conditions and arising from a poor structural diversity of the starting compounds. To date, in the literature there are no precedents of this kind.

### SUMMARY OF THE INVENTION

The object of the invention is the development of new compositions based on polyphenols and polysaccharides and methods for preparing thereof.

The present invention relates to an antimicrobial, preferably antiviral composition comprising a biologically effective amount of an active component, wherein the active component is a product of co-treatment of an aqueous and/or aqueous-organic solution of polyphenol “D” and an aqueous and/or aqueous-organic solution of polysaccharide “P” with organic and/or inorganic acids or bases and/or organic and/or inorganic salts at a pH of 0.1 to 14 and at a temperature of from the solvent freezing temperature to the solvent boiling point until a content of carbonyl and/or hydroxyl groups in the polysaccharide reaches 99.99 to 0% of the original content and until a conversion of the starting polyphenol to polyphenols with a molecular weight of 100 to 2000 atomic mass units (AMU) reaches 0.1 to 100%, wherein the mass ratio of the polyphenol “D” to the polysaccharide “P” is from 1000:1 to 1:1000; optionally followed by neutralization of the reaction mixture with organic and/or inorganic acids or bases to a pH of 1 to 13, preferably 7 to 10, followed by isolation and purification of the resulting product;

wherein the polyphenol “D” is from 1 to 100 compounds, each of which has a molecular weight of from 100 to 2000 AMU, from 2 to 40 phenolic groups, and from 0 to 20 functional groups other than phenolic ones, which can be pretreated with organic and/or inorganic acids or bases and/or organic and/or inorganic salts in an aqueous and/or aqueous-organic solution containing from 0 to 100% organic solvents, at a pH of from 0.1 to 14 until a conversion of the starting polyphenol to polyphenols with a molecular weight of 100 to 2000 AMU reaches 0.1 to 100%;

preferably polyphenol “D” is 1 to 50 compounds, each of which contains 2 to 6 phenolic groups and 1 to 12 functional groups other than phenolic ones with a molecular weight of from 120 to 700 AMU;

most preferably, polyphenol “D” is 1 to 40 compounds, each of which contains 2 to 6 phenolic groups and 1 to 8 functional groups other than phenolic ones with a molecular weight of from 300 to 550 AMU;

wherein the polysaccharide “P” contains 1 to 10 polysaccharide chains, preferably one polysaccharide chain, each having a weight-average molecular weight of 0.4 to 3000 kDa, preferably from 1 to 500 kDa, most preferably from 1 to 50 kDa, consisting of covalently bound units of the following composition:

[A][B][C];

wherein

“A” represents units in a polysaccharide structure of the following general formula:

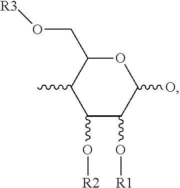

wherein

R1, R2, and R3 independently are H, polysaccharide “P”, (R5CH)ₙCOOR4;

R4 is H, Li, Na, and K; preferably R4 is H, Na;

R5 is H, linear or branched C₁-C₁₀ alkyl; preferably R5 is H;

n is from 0 to 10; preferably n is from 1 to 2;

“B” is oxidized units in the polysaccharide structure, having a molecular weight of from 15 to 500 Da, containing 1 to 6 functional groups capable of undergoing nucleophilic addition reactions;

preferably, “B” is oxidized units in the polysaccharide structure, having a molecular weight of from 150 to 300 Da, containing 1 to 3 functional groups capable of undergoing nucleophilic addition reactions;

“C” is a product of transformation of oxidized units under conditions of the treatment of an aqueous and/or aqueous-organic solution of the polysaccharide “P” with organic and/or inorganic acids or bases and/or organic and/or inorganic salts at a pH of 0.1 to 14 and at a temperature of from the solvent freezing point to the solvent boiling point, until a content of carbonyl and/or hydroxyl groups in the polysaccharide reaches 99.99 to 0% of the original content;

preferably “C” is a product of transformation of oxidized units under conditions of the treatment of an aqueous and/or aqueous-organic solution of the polysaccharide “P” with organic and/or inorganic acids or bases and/or organic and/or inorganic salts at a pH of 0.1 to 14 and at a temperature of from the solvent freezing point to the solvent boiling point, until a content of carbonyl and/or hydroxyl groups in the polysaccharide reaches 95 to 20% of the original content;

and

wherein said polysaccharide can be pretreated with organic and/or inorganic acids or bases and/or organic and/or inorganic salts in an aqueous-organic medium containing 0 to 100% of an organic solvent, at a pH of 1 to 14 and at a temperature of from the solvent freezing temperature to the solvent boiling point until a content of carbonyl and/or hydroxyl groups in the polysaccharide reaches 99.99 to 0% of the original content, optionally with additional steps of purification and/or desalting and/or fractionation;

and pharmaceutically acceptable excipients.

In another aspect, the present invention relates to an antiviral and antimicrobial composition comprising:

a biologically effective amount of an active component, wherein the active component is polysaccharide “P” that contains 1 to 10 polysaccharide chains, preferably one polysaccharide chain, each having a weight-average molecular weight of 0.4 to 3000 kDa, preferably of 1 to 500 kDa, most preferably of 1 to 50 kDa, consisting of covalently linked units of the following structure:

[A][B][C];

wherein

“A” represents units in a polysaccharide structure of the following general formula:

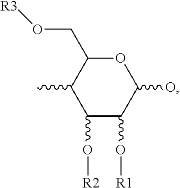

wherein

R1, R2, and R3 independently are H, polysaccharide “P”, (R5CH)ₙCOOR4;

R4 is H, Li, Na, and K; preferably R4 is H, Na;

R5 is H, linear or branched C₁-C₁₀ alkyl; preferably R5 is H;

n is 0 to 10, preferably n is 1 to 2;

“B” is oxidized units in the polysaccharide structure, having a molecular weight of from 15 to 500 Da, containing 1 to 6 functional groups capable of undergoing nucleophilic addition reactions;

preferably, “B” is oxidized units in the polysaccharide structure, having a molecular weight of from 150 to 300 Da, containing 1 to 3 functional groups capable of undergoing nucleophilic addition reactions;

“C” is a product of transformation of oxidized units under conditions of the treatment of an aqueous and/or aqueous-organic solution of the polysaccharide “P” with organic and/or inorganic acids or bases and/or organic and/or inorganic salts at a pH of 0.1 to 14 and at a temperature of from the solvent freezing point to the solvent boiling point until a content of carbonyl and/or hydroxyl groups in the polysaccharide reaches 99.99 to 0% of the original content;

preferably “C” is a product of transformation of oxidized units under conditions of the treatment of an aqueous and/or aqueous-organic solution of the polysaccharide “P” with organic and/or inorganic acids or bases, and/or organic and/or inorganic salts at a pH of 0.1 to 14 and at a temperature of from the solvent freezing point to the solvent boiling point until a content of carbonyl and/or hydroxyl groups in the polysaccharide reaches 95 to 20% of the original content;

and

wherein said polysaccharide can be pretreated with organic and/or inorganic acids or bases and/or organic and/or inorganic salts in an aqueous-organic medium containing 0 to 100% of an organic solvent, at a pH of 1 to 14 and at a temperature of from the solvent freezing temperature to the solvent boiling point until a content of carbonyl and/or hydroxyl groups in the polysaccharide reaches 99.99 to 0% of the original content, optionally with additional steps of purification and/or desalting and/or fractionation;

and pharmaceutically acceptable excipients.

Another object of the present invention is an antimicrobial combination comprising one of the above compositions and at least one extract of plant materials, wherein the combination has at least one of antioxidant, antibacterial, immunostimulating, antimicrobial, antitumor, and anti-inflammatory activities, wherein the content of the extract in the combination is from 0.01 to 99.99%, preferably from 1.0 to 50.0%, most preferably from 1.0 to 10.0%.

In a preferred embodiment, the plant extract is an aqueous, alcoholic, oily or organic extract from the following plant materials: astragalus (roots), acerola, artichoke (leaves), angelica, black elderberry, hawthorn (fruits, leaves, and flowers), birch (buds), valerian (roots and rootstocks), grape seeds, hibiscus, elecampane, oak, ginseng, green tea, ginger, strawberry (leaves), cranberry, white willow (bark), calendula, aspen (bark), grapefruit, watercress, burdock, raspberry (fruits), juniper, bitter melon, peppermint, blueberry, motherwort herb, milk thistle, pharmaceutical chamomile, sabelnik, soybean (beans), licorice (roots), sea buckthorn (berries), fennels, horseradish, thyme, garlic, sage, eleutherococcus, purple echinacea, or combinations thereof.

Another object of the present invention is an antimicrobial combination comprising one of the above compositions and at least one antimicrobial pharmaceutical substance, wherein the content of the substance in the combination is from 0.0000001 to 99.9999999%, preferably from 0.0000001 to 10.0%, most preferably from 0.0001 to 5.0%.

Arbidol, Oseltamivir, and Rimantadine are preferably used as the pharmaceutical substance.

Another object of the invention is a method for preparing the above compositions, comprising:
a step of pretreatment of polyphenol “D” with organic and inorganic acids or bases, and/or organic and/or inorganic salts in an aqueous-organic medium containing from 0 to 100% of an organic solvent, at a pH in the range of 0.1 to 14 and at a temperature of from the solvent freezing temperature to the solvent boiling point until a conversion of the starting polyphenol to polyphenols with a molecular weight of 100 to 2000 AMU reaches 0.01 to 100%;

and/or a pretreatment step of the polysaccharide “P” with organic and/or inorganic acids or bases and/or organic and/or inorganic salts in an aqueous-organic medium containing from 0 to 100% of an organic solvent, at a pH in the range of 1 to 14 and at a temperature of from the solvent freezing temperature to the solvent boiling point until a content of carbonyl and/or hydroxyl groups in the polysaccharide reaches 99.99 to 0% of the original content, optionally with additional steps of purification and/or desalting and/or fractionation;
a step of treating an aqueous and/or aqueous-organic solution of the polyphenol “D” and an aqueous and/or aqueous-organic solution of the polysaccharide “P” with organic and/or inorganic acids or bases and/or with organic and/or inorganic salts at a pH of 0.1 to 14 and a temperature of from the solvent freezing point to the solvent boiling point until a content of carbonyl and/or hydroxyl groups in the polysaccharide reaches 99.99 to 0% of the original content and/or until a conversion of the starting polyphenol to polyphenols with a molecular weight of from 100 to 2000 AMU reaches 0.01 to 100%, wherein a mass ratio of polyphenol to polysaccharide is from 1000:1 to 0:1000;
a subsequent step of isolation and purification of a resulting product; and
a step of combining the resulting product with pharmaceutically acceptable excipients.

The method for preparing the above compositions may include an optional step of neutralization of the reaction mixture with organic and/or inorganic acids or bases to a pH of 1 to 13, preferably 7 to 10.

The polyphenol is preferably 1 to 10 compounds, each of which contains 2 to 6 phenolic groups, 2 to 12 functional groups other than phenolic ones with a molecular weight of from 120 to 700 AMU, most preferably apogossypol, gossypolone, gossindan, apogossypolone, 1,1′,6,6′,7,7′-hexahydroxy-5,5′-diisopropyl-3,3′-dimethyl-2,2′-binaphthalene-8-carbaldehyde, 6,6′,7,7′-tetrahydroxy-5,5′-diisopropyl-3,3 dimethyl-1,1′,4,4′-tetraoxo-1,1′,4,4′-tetrahydro-2,2′-binaphthalene-8-carbaldehyde, ethyl [(8-formyl-1,1′,6,6′,7′-pentahydroxy-5,5′-diisopropyl-3,3′-dimethyl-2,2′-binaphthalene-7-yl)oxy]acetate, gossypol, gossypol acetic acid.

At the pretreatment step, the conversion of the starting polyphenol to polyphenols with a molecular weight of from 100 to 2000 AMU is preferably from 50 to 100%, most preferably from 80 to 100%.

The polysaccharide is preferably cellulose, carboxymethyl cellulose, dextran, carboxymethyl dextran, dialdehyde carboxymethyl cellulose, dialdehyde dextran, dialdehyde cellulose, dialdehyde carboxymethyl dextran, starch, dialdehyde starch, dextrin, dialdehyde dextrin, and products of their conversion in aqueous and\or aqueous-organic solutions at a pH of 0, 1 to 14.

In the step of pre-treating the polysaccharide, the content of carbonyl and/or hydroxyl groups in the polysaccharide is preferably from 95.0 to 20.0% of the original content.

The pretreatment step is carried out at a pH of preferably from 7 to 14, most preferably from 10 to 14.

The pretreatment is preferably carried out at a temperature of from 10 to 60° C.

In the pretreatment step, the organic solvent is preferably acetone, ethyl alcohol, isopropyl alcohol, 1,4-dioxane, tetrahydrofuran.

In the treatment step, the conversion of the starting polyphenol to polyphenols with a molecular weight of from 100 to 2000 AMU is preferably from 50 to 100%, most preferably from 80 to 100%.

In the treatment step, the content of carbonyl and/or hydroxyl groups in the polysaccharide is preferably from 95.0 to 20.0% of the original content.

The treatment step is carried out at a pH of preferably from 7 to 14, most preferably from 10 to 14.

The treatment step is carried out at a temperature of preferably from 10 to 60° C., most preferably from 18 to 55° C.

In the treatment step, the organic solvent is preferably acetone, ethyl alcohol, isopropyl alcohol, 1,4-dioxane, tetrahydrofuran.

The steps pretreatment of the polyphenol “D” and polysaccharide “P” are preferably performed simultaneously in the same reactor.

Another object of the present invention is an antimicrobial agent containing one of the above compositions. The antimicrobial agent according to the present invention is effective against influenza, herpes, hepatitis, and HIV viruses, respiratory viral infections and bacterial infections.

Another object of the present invention is a dietary supplement providing one or more effects, preferably antioxidant, antibacterial, immunostimulating, antimicrobial, antitumor, anti-inflammatory effects, wherein the dietary supplement contains a combination according to the present invention.

Another object of the present invention is an antimicrobial agent containing a combination according to the present invention.

### DESCRIPTION OF DRAWINGS

FIG. 1 shows chromatograms of the compositions prepared by modifying the introduced polyphenol; detection was made at 254 nm.

FIG. 2 shows chromatogram of the composition prepared without the introduction of polyphenols; detection was made at 254 nm.

FIG. 3 shows DOSY spectra for the samples according to example 9.

FIG. 4 shows the IR spectrum of the composition (example 8) in comparison with the starting polymer, which are prepared by the ATR method.

### DETAILED DESCRIPTION OF THE INVENTION

In the context of the present invention:

the term “co-treatment” means the simultaneous presence of two or more starting compounds in one reaction mixture until the termination of the reaction;

the term “pretreatment” means the presence of one starting compound (polysaccharide or polyphenol) in the indicated physicochemical conditions until achieving desired structural changes, prior to the introduction of a second compound (polyphenol or polysaccharide);

the term “polyphenol” means a class of chemical compounds characterized by the presence of more than one phenolic group, preferably 2 to 6 phenolic groups with a molecular weight of from 100 to 2000 AMU, preferably from 100 to 700 AMU, containing from 0 to 20 functional groups other than phenolic ones, preferably from 0 to 8 functional groups other than phenolic ones, in particular, gossypol, apogossypol, gossypolone, gossindan, apogossypolone, 1,1′,6,6′,7,7′-hexahydroxy-5,5′-diisopropyl-3,3′-dimethyl-2,2′-binaphthalene-8-carbaldehyde, 6,6′,7,7′-tetrahydroxy-5,5′-diisopropyl-3,3′-dimethyl-1,1′,4,4′-tetraoxo-1,1′,4,4′-tetrahydro-2,2′-binaphthalene-8-carbaldehyde, gallic acid, epigallocatechin, gallol;

the term “aqueous solution” means a solution with a solute concentration of from 0.00001 to 99.999 wt. %, wherein the solvent is water;

the term “aqueous-organic solution” means a solution with a solute concentration of from 0.00001 to 99.999 wt. %, wherein the solvent is a homogeneous mixture of water and an organic solvent at a mass ratio of from 100:0 to 0:100, in particular, a mixture of water with ethanol, a mixture of water with acetone, a mixture of water with isopropanol, a mixture of water with dioxane;

the term “organic acid” means an organic compound exhibiting acidic properties, in particular, formic acid, acetic acid, oxalic acid, p-toluenesulfonic acid, citric acid, tartaric acid;

the term “inorganic acid” means an inorganic compound exhibiting acidic properties, in particular, hydrochloric acid, sulfuric acid, phosphoric acid, perchloric acid, carbonic acid, nitric acid;

the term “organic base” means any organic compound capable of accepting positively charged ions, in particular, triethylamine, 4-methylmorpholine, N-ethyldiisopropylamine, potassium tert-butylate, sodium ethylate;

the term “inorganic base” means any inorganic compound capable of accepting positively charged ions, in particular, sodium carbonate, potassium hydroxide, sodium acetate, sodium hydroxide, sodium bicarbonate, cesium carbonate, potassium carbonate;

the term “organic salt” means any organic compound that dissociates in aqueous solutions into cations and anions of organic acid residues, in particular, oxalates, carboxylates, alcoholates, acetates, phenolates, ascorbates, tartrates, citrates, pyridinium salts;

the term “inorganic salt” means any inorganic compound that dissociates in aqueous solutions into cations and anions of acid residues, in particular, chlorides, sulfates, carbonates;

the term “organic solvent” means any organic compound capable of dissolving various substances, in particular, aliphatic and aromatic hydrocarbons and their halogen derivatives, alcohols, ethers and esters, ketones;

the term “functional group” means a combination of atoms, which determines the characteristic chemical properties of a given class of compounds, in particular, hydroxyl, carbonyl, carboxyl, alkyl, aryl, and other groups;

the term “phenolic group” means a hydroxyl group bound to a carbon atom of an aromatic or heteroaromatic ring;

the term “polysaccharide” means a monosaccharide polycondensation product comprising monosaccharide units linked to each other through any oxygen atom or a product of synthetic modification of carbohydrates, in particular, cellulose, carboxymethyl cellulose, dextran, carboxymethyl dextran, dialdehyde carboxymethyl cellulose, dialdehyde dextran, dialdehyde cellulose, dialdehyde carboxymethyl dextran, starch, dialdehyde starch, dextrin, dialdehyde dextrin, and products of their transformation in aqueous and/or aqueous-organic solutions at a pH of from 0.1 to 14;

the term “covalently linked units” means units of one or several polysaccharide chains linked to each other by a covalent bond through any atom of one link with the involvement of any functional group of another unit, in particular, hydroxyl, carbonyl, hemiacetal, carboxyl, and others;

the term “polysaccharide chain” means a monosaccharide polycondensation product containing monosaccharide units linked to each other through any oxygen atom or a product of synthetic modification of carbohydrates, containing structurally related polysaccharide fragments of one type with different number of units and molecular weight distribution, in particular, cellulose, carboxymethyl cellulose, dextran, carboxymethyl dextran, dialdehyde carboxymethyl cellulose, dialdehyde dextran, dialdehyde cellulose, dialdehyde carboxymethyl dextran, starch, dialdehyde starch, dextrin, starch, dialdehyde dextrin, and products of their transformation in aqueous and/or aqueous-organic solutions at a pH of from 0.1 to 14;

the term “oxidized unit” means one or more products of the transformation of a polysaccharide unit under the action of oxidizing agents, wherein the transformation is accompanied by a change in the structure of the polysaccharide unit and by the appearance of new functional groups presented in free carbonyl, hemi-aldal, hemiacetal, acetal forms, in particular,

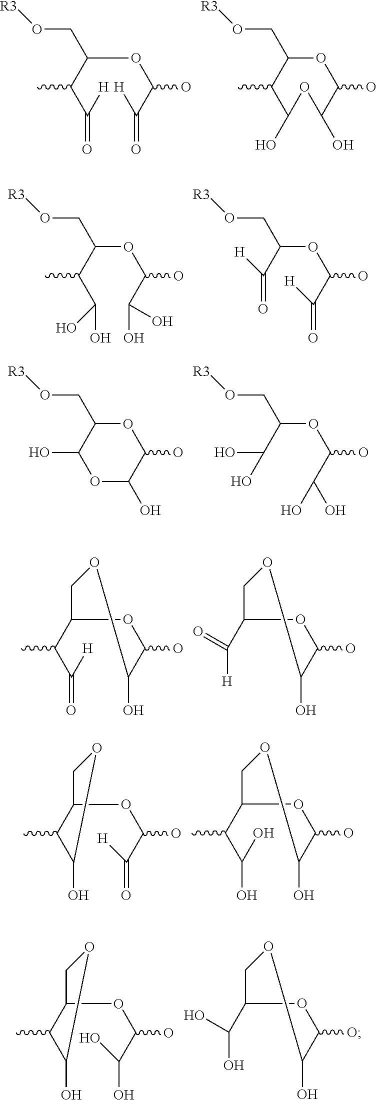

the term “free carbonyl form” means a product of the transformation of a polysaccharide unit under the action of oxidizing agents, wherein the product contains more than one carbonyl group, in particular,

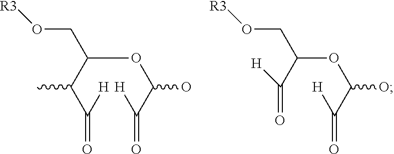

the term “hemi-aldal form” means a product of the transformation of a polysaccharide unit under the action of oxidizing agents, in which the aldehyde groups in the product are in a state in which they are bound with one or more water molecules, in particular,

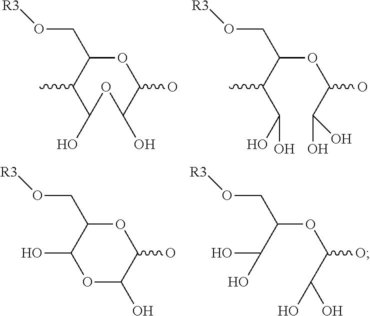

the term “hemiacetal form” means a product of the transformation of a polysaccharide unit, in which at least one aldehyde group is bound to a substituted oxygen atom of the polysaccharide unit or other compounds, in particular,

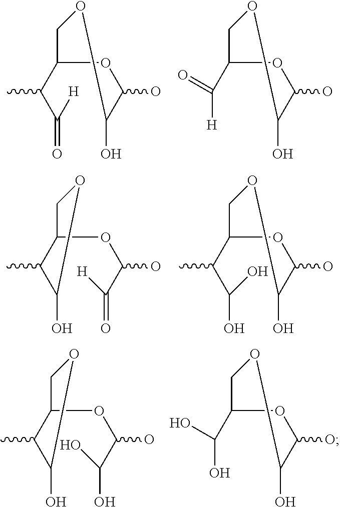

the term “acetal form” means a product of the transformation of a polysaccharide unit, in which at least one aldehyde group is bound to two substituted oxygen atoms of the polysaccharide unit or other compounds;

the term “substituted oxygen atom” means any ROH structure in which substituent R is not hydrogen or oxygen;

the term “pharmaceutically acceptable excipient” means a compound that is approved for use in the pharmaceutical industry to prepare finished dosage forms and that is not an active ingredient but can influence both the biological efficiency of the active ingredient and the physical properties of the finished dosage form;

the term “combination” means a homogeneous or almost homogeneous mixture of components, having a biological effect different from the additive effect of the components thereof;

the term “extract of plant materials” means a substance obtained by extracting and concentrating base material of plant origin.

Test Methods

Nuclear Magnetic Resonance (NMR) Spectroscopy

The method was used to determine the molecular structure of substances.

For obtaining spectral data, an accurately weighed sample (15-30 mg) was dissolved in 50 mg of D₂O with a pH of 10 to 11 (adjusted with Na₂CO₃ or NaOH), and 50 μl of a TSP solution in D₂O at a concentration of 1 mg/ml (the amount of the added TSP was 50 μg) were added to the solution. The solutions were transferred into an NMR vial (d=5 mm) and placed in an NMR spectrometer. The magnetic field uniformity was adjusted. Spectra were recorded. Signals were set according to a TSP chemical shift equal to 0 ppm or according to residual protons of the solvent signal.

DOSY spectra were recorded under the following conditions: the number of points per spectrum was 32K; the delay between pulses was 15 s; the pulse angle was 90°; the number of spectrum accumulations was 64, gradient containing 16 points was from 3 mT/m to 0.4 T/m, and diffusion time was 0.1 s.

High Performance Liquid Chromatography with UV Spectrophotometric and Mass Spectrometric Detection (HPLC-UV-MS)

The method was used to determine the qualitative and quantitative content of the compositions. The analysis was performed on an Agilent 1260 Liquid Chromatography System with sequential detection on diode-array and mass-selective detectors. Separation was made on a Zorbax Eclipse Plus C18 chromatographic column (Agilent) by using as eluents a 0.1% solution of formic acid in water and acetonitrile in a gradient elution mode. Ionization in the used single-quadrupole mass spectrometry detector was carried out by an electrospray method; the detection was performed in a negative-ion reflectron mode. The detection in a diode-matrix detector was performed at a wavelength of 254 nm, and the UV spectrum was recorded in the range from 200 to 400 nm.

Fourier Transform Infrared Spectroscopy (IR)

IR spectroscopy was used to confirm the structure of the resulting compounds. Spectra were recorded on a Spectrum 65 spectrometer (Perkin Elmer) in a potassium bromide disk in the range of from 4000 to 400 cm⁻¹, and by the ATR method in the range of from 4000 to 650 cm⁻¹.

Spectrophotometry in Ultraviolet and Visible Spectral Regions (UV-Vis)

The method of spectrophotometry was used to confirm the identity of the resulting compounds. Spectra were recorded on a Lambda 25 spectrophotometer (Perkin Elmer) in 1 cm thick quartz cells in the range of 220 to 700 cm⁻¹.

Potentiometric Acid-Base Titration

The method of potentiometric acid-base titration was used to determine the number of carbonyl groups in samples.

The determination was performed by an oxime method, which is based on the reaction of aldehyde groups with hydroxylamine hydrochloride, which results in the formation of hydrochloric acid that is titrated with a sodium hydroxide solution.

Gel Permeation Chromatography (GPC)

The method of gel permeation chromatography was used to determine the molecular weight and molecular weight characteristics of polymer compositions and raw materials. The analysis was performed on a Waters Liquid chromatography system with sequential detection on refractometric and diode-array detectors. Separation was performed on Ultrahydrogel 1000 and Ultrahydrogel 120 columns (300×7.8 mm) connected in series and filled with hydroxylated polymethacrylate gel with a pore size of 1000 and 120 Å, respectively (Waters), using a 0.05 M phosphate buffer solution (pH=7.0) as an eluent in the isocratic elution mode.

The column was calibrated by using dextran standards with weight-average molecular weights (Mw) of 9900, 16230, 41100, 60300, 129000, 214800, 456800.

The calibration curves were approximated by a third-order polynomial. The molecular mass characteristics of polymer were calculated using universal calibration and software “Breeze 2”.

### Example 1

A 200 ml beaker was filled with 27.6 g of water, and then 60 g of a NaOH solution (34.8%) were added thereto. After that, 60 g of dialdehyde carboxymethyl cellulose (hereinafter referred to as DACMC) with a carbonyl group content of 0.94 mmol/g were added with stirring, while maintaining the temperature not higher than 58° C. The reaction mass was stirred for 3 hours at room temperature, then the stirring was stopped, and the reaction mass was aged for 18 hours until the content of carbonyl groups reached 81.4% of the original polymer. On the next day, the reaction mass was acidified with 10 g of sodium bicarbonate, stirred for 30 minutes, precipitated from acetone and dried. The yield of the resulting composition was 76.8 g.

### Example 2

A 200 ml beaker was filled with 70 g of a NaOH solution (20%). After that, 7 g of DACMC with a carbonyl group content of 2.3 mmol/g were added with stirring, and the reaction mass was stirred for 60 minutes at 25° C. until the content of carbonyl groups in the polysaccharide reached 26.5% of the original content, after which 23 g of sodium bicarbonate were added to the reaction mass and stirred for 30 minutes. Further, the reaction mass was precipitated from acetoneand dried in a dry-heat oven. The yield of the resulting composition was 39.2 g.

### Example 3

A 200 ml beaker was filled with 70 g of a Na₂CO₃ solution (20%). After that, 7 g of DACMC with a carbonyl group content of 2.3 mmol/g were added with stirring, and the reaction mass was stirred for 180 minutes at 25° C. until the content of carbonyl groups in the polysaccharide reached 62.2% of the original content. Further, the reaction mass was precipitated from acetoneand dried in a dry-heat oven. The yield of the resulting composition was 18.8 g.

### Example 4

A 200 ml beaker was filled with 20 g of water, and then 1 g of a NaOH solution (10%) was added thereto. After that, 1 g of dialdehyde dextran (hereinafter referred to as DAD) with a carbonyl group content of 1.60 mmol/g was added with stirring, while maintaining the temperature not higher than 58° C. The reaction mass was stirred for 3 hours at room temperature, then the stirring was stopped, and the reaction mass was aged for 18 hours until the content of carbonyl groups reached 46.9% of the original polymer. On the next day, the reaction mass was acidified with 0.12 g of sodium bicarbonate, stirred for 30 minutes, lyophilized and dried. The yield of the resulting composition was 1.0 g.

### Example 5

A 200 ml beaker was filled with 20 g of water, and then 1 g of a NaOH solution (34.8%) was added thereto. After that, 1 g of DAD with a carbonyl group content of 1.60 mmol/g was added with stirring, while maintaining the temperature not higher than 58° C. The reaction mass was stirred for 3 hours at room temperature, then the stirring was stopped, and the reaction mass was aged for 18 hours until the content of carbonyl groups reached 66.2% of the original polymer. On the next day, the reaction mass was acidified with 0.12 g of sodium bicarbonate, stirred for 30 minutes, lyophilized and dried. The yield of the resulting composition was 1.4 g.

### Example 6

A 200 ml beaker was filled with 9.2 g of water and 20 g of a NaOH solution (34.8%). After that, 20 g of DACMC with a carbonyl group content of 0.94 mmol/g were added with stirring, and the reaction mass was stirred for 20 minutes at 25° C. until the content of carbonyl groups in the polysaccharide reached 81.4% of the original content. After that 0.02 g of hemigossypol in 2 ml of acetone was added to the reaction mass. Further, the reaction mass was stirred for 20 min at 25° C. until the conversion of the starting polyphenol to polyphenols having a molecular weight of 100 to 2000 AMU reached 100% and the carbonyl group content in the polysaccharide reached 81.4% of the original content. The reaction mass was then lyophilized. The yield of the resulting composition was 20.2 g.

### Example 7

A 200 ml beaker was filled with 9.2 g of water and 20 g of a NaOH solution (34.8%). After that, 20 g of DACMC with a carbonyl group content of 0.94 mmol/g were added with stirring, and the reaction mass was stirred for 20 minutes at 25° C. until the content of carbonyl groups in the polysaccharide reached 81.4% of the original content. After that 0.1 g of hemigossypol in 5 ml of acetone was added thereto. Further, the reaction mass was stirred for 20 min at 25° C. until the conversion of the starting polyphenol to polyphenols having a molecular weight of 100 to 2000 AMU reached 86.4% and the carbonyl group content in the polysaccharide reached 81.4% of the original content. The reaction mass was then lyophilized. The yield of the resulting composition was 20.5 g.

### Example 8

A 200 ml beaker was filled with 10.0 g of water and 24 g of a NaOH solution (34.8%). After that, 24 g of DACMC with a carbonyl group content of 0.94 mmol/g were added with stirring, and the reaction mass was stirred for 20 minutes, while maintaining the temperature not higher than 58° C., until the content of carbonyl groups in the polysaccharide reached 89.36% of the original content. A solution of gossypol was concurrently prepared by dissolving 0.4 g of gossypol acetic acid in 25 ml of acetone, 2.4 ml of a NaOH solution (34.8%), and 10.0 ml of water for 10 min at 25° C. Then, the polyphenol solution was added to the polymer solution. Further, the reaction mass was stirred for 10 min at 37° C. and then for 60 min at 20° C. until the conversion of the starting polyphenol to polyphenols having a molecular weight of 100 to 2000 AMU reached 100% and the carbonyl group content in the polysaccharide reached 89.36% of the original content. Then, the pH of the reaction mixture was adjusted to 11 by adding NaHCO₃, and the product was precipitated from acetone. The yield of the resulting composition was 28.7 g.

### Example 9

A 200 ml beaker was filled with 10.0 g of water and 24 g of a NaOH solution (34.8%). After that, 24 g of DACMC with a carbonyl group content of 0.94 mmol/g were added with stirring, and the reaction mass was stirred for 20 minutes, while maintaining the temperature not higher than 58° C., until the content of carbonyl groups in the polysaccharide reached 93.61% of the original content. A solution of gossypol was concurrently prepared by dissolving 1.6 g of gossypol acetic acid in 25 ml of acetone, 2.4 ml of a NaOH solution (34.8%), and 10.0 ml of water for 10 min at 25° C. Then, the polyphenol solution was added to the polymer solution. Further, the reaction mass was stirred for 10 min at 37° C. and then for 60 min at 20° C. until the conversion of the starting polyphenol to polyphenols having a molecular weight of 100 to 2000 AMU reached 100% and the carbonyl group content in the polysaccharide reached 93.61% of the original content. Then, the pH of the reaction mixture was adjusted to 11 by adding NaHCO₃, and the product was precipitated from acetone. The yield of the resulting composition was 28.7 g.

### Example 10

A 500 ml beaker was filled with 200 g of a NaOH solution of (20%). After that, 20 g of DACMC with a carbonyl group content of 0.94 mmol/g and 0.2 g of gossypol were added with stirring. The reaction mass was stirred for 72 hours at 25° C. until the content of carbonyl groups in the polysaccharide reached 24.5% of the original content and the conversion of the starting polyphenol to polyphenols having molecular weight of 100 to 2000 AMU reached 100%. After that, 75 g of sodium bicarbonate were added to the reaction mass and stirred for 30 min. The reaction mass was then lyophilized. The yield of the resulting composition was 45.4 g.

### Example 11

A 500 ml beaker was filled with 200 g of a NaOH solution (20%). 20 g of DACMC with a carbonyl group content of 0.94 mmol/g and 0.6 g of gossypol were then added with stirring. The reaction mass was stirred for 72 hours at 25° C. until the content of carbonyl groups in the polysaccharide reached 34.0% of the original content and the conversion of the starting polyphenol to polyphenols having molecular weight of 100 to 2000 AMU reached 100%. After that, 75 g of sodium bicarbonate were added to the reaction mass and stirred for 30 min. The reaction mass was then lyophilized. The yield of the resulting composition was 49.5 g.

### Example 12

A 500 ml beaker was filled with 200 g of a NaOH solution (20%). After that, 20 g of DACMC with a carbonyl group content of 0.94 mmol/g and 1 g of gossypol were added with stirring. The reaction mass was stirred for 72 hours at 25° C. until the content of carbonyl groups in the polysaccharide reached 34.0% of the original content and the conversion of the starting polyphenol to polyphenols having molecular weight of 100 to 2000 AMU reached 100.0%. After that, 75 g of sodium bicarbonate were added to the reaction mass and stirred for 30 min. The reaction mass was then lyophilized. The yield of the resulting combination was 39.5 g.

### Example 13

A 200 ml beaker was filled with 27.6 g of water, and then 60 g of a NaOH solution (34.8%) were added thereto. After that, 60 g of DACMC with a carbonyl group content of 0.94 mmol/g were added with stirring, while maintaining the temperature not higher than 58° C. The reaction mass was stirred for 3 hours at room temperature, then the stirring was stopped, and the reaction mass was aged for 18 hours until the content of carbonyl groups reached 81.4% of the original polymer. On the next day, the reaction mass was acidified with 10 g of sodium bicarbonate, stirred for 30 minutes, isolated into acetone, and dried. The resulting substance was loaded into an ultrafiltration unit, and ultrafiltration was carried out on a 10-kDa membrane, providing a polymer intermediate with a carbonyl group content of 0.85 mmol/g.

A 50 ml beaker was filled with 20 g of water. Then, 0.5 g of the polymer intermediate with a carbonyl group content of 0.85 mmol/g was added with stirring, and the reaction mass was stirred for 10 minutes at 25° C., after which 1 ml of an oseltamivir phosphate solution (10⁻³ g), preliminarily aged for 5 minutes at 25° C. in water, was added thereto The reaction mass was stirred for 30 min at 25° C. The reaction mass was then lyophilized. The yield of the resulting combination is 0.5 g.

### Example 14

A 200 ml beaker was filled with 27.6 g of water, and then 60 g of a NaOH solution (34.8%) were added thereto. After that, 60 g of DACMC with a carbonyl group content of 0.94 mmol/g were added with stirring, while maintaining the temperature not higher than 58° C. The reaction mass was stirred for 3 hours at room temperature, then the stirring was stopped, and the reaction mass was aged for 18 hours until the content of carbonyl groups reached 81.4% of the original polymer. On the next day, the reaction mass was acidified with 10 g of sodium bicarbonate, stirred for 30 minutes, isolated into acetone, and dried. The polymer intermediate with a carbonyl group content of 0.79 mmol/g was obtained.

A 100 ml beaker was filled with 50 ml of acetone. Then, 10 g of the polymer intermediate with a carbonyl group content of 0.79 mmol/g were added with stirring, and the reaction mass was stirred for 5 min at 25° C. After that, 0.3 g of echinacea extract in 3 ml of acetone was added to the reaction mass. The reaction mass was stirred for 30 min at 25° C. The reaction mass was evaporated to dryness on a rotary evaporator and dried in a dry-heat oven at 40° C. The yield of the resulting combination was 10 g.

### Example 15

A 200 ml beaker was filled with 27.6 g of water, and then 60 g of a NaOH solution (34.8%) were added thereto. After that, 60 g of DACMC with a carbonyl group content of 0.94 mmol/g were added with stirring, while maintaining the temperature not higher than 58° C. The reaction mass was stirred for 3 hours at room temperature, then the stirring was stopped, and the reaction mass was aged for 18 hours until the content of carbonyl groups reached 81.4% of the original polymer. On the next day, the reaction mass was acidified with 10 g of sodium bicarbonate, stirred for 30 minutes, isolated into acetone, and dried. The polymer intermediate with a carbonyl group content of 0.79 mmol/g was obtained.

A 100 ml beaker was filled with 50 ml of acetone. Then, 10 g of the polymer intermediate with a carbonyl group content of 0.79 mmol/g were added with stirring, and the reaction mass was stirred for 5 min at 25° C. After that, 0.3 g of licorice extract in 3 ml of acetone was added to the reaction mass. The reaction mass was stirred for 30 min at 25° C. The reaction mass was evaporated to dryness on a rotary evaporator and dried in a dry-heat oven at 40° C. The yield of the resulting combination is 10 g.

### Example 16

A 200 ml beaker was filled with 27.6 g of water, and then 60 g of a NaOH solution (34.8%) were added thereto. After that, 60 g of DACMC with a carbonyl group content of 0.94 mmol/g were added with stirring, while maintaining the temperature not higher than 58° C. The reaction mass was stirred for 3 hours at room temperature, then the stirring was stopped, and the reaction mass was aged for 18 hours until the content of carbonyl groups reached 81.4% of the original polymer. On the next day, the reaction mass was acidified with 10 g of sodium bicarbonate, stirred for 30 minutes, isolated into acetone, and dried. The polymer intermediate with a carbonyl group content of 0.79 mmol/g was obtained.

A 100 ml beaker was filled with 50 ml of acetone. Then, 10 g of the polymer intermediate with a carbonyl group content of 0.79 mmol/g were added with stirring, and the reaction mass was stirred at 25° C. for 5 min. After that, 0.3 g of ginger extract in 3 ml of acetone was added to the reaction mass. The reaction mass was stirred for 30 min at 25° C. The reaction mass was evaporated to dryness on a rotary evaporator and dried in a dry-heat oven at 40° C. The yield of the resulting combination is 10 g.

### Example 17

A 200 ml beaker was filled with 27.6 g of water, and then 60 g of a NaOH solution (34.8%) were added thereto. After that, 60 g of DACMC with a carbonyl group content of 0.94 mmol/g were added with stirring, while maintaining the temperature not higher than 58° C. The reaction mass was stirred for 3 hours at room temperature, then the stirring was stopped, and the reaction mass was aged for 18 hours until the content of carbonyl groups reached 81.4% of the original polymer. On the next day, the reaction mass was acidified with 10 g of sodium bicarbonate, stirred for 30 minutes, precipitated from acetone, and dried. The polymer intermediate with a carbonyl group content of 0.79 mmol/g was obtained.

A 100 ml beaker was filled with 50 ml of acetone. Then, 10 g of the polymer intermediate with a carbonyl group content of 0.79 mmol/g were added with stirring, and the reaction mass was stirred for 5 min at 25° C. After that, 0.3 g of elecampane extract in 3 ml of acetone was added to the reaction mass. The reaction mass was stirred for 30 min at 25° C. The reaction mass was evaporated to dryness on a rotary evaporator and dried in a dry-heat oven at 40° C. The yield of the resulting combination is 10 g.

TABLE 1

| | Composition | | |
|---|---|---|---|
| | Polymer | Polyphenol | Salt |
| | content, | content, | content |
| Sample | wt. % | wt. % | wt. % |
| According to example 1 | 84.1 | 0 | 15.9 |
| According to example 2 | 37.6 | 0 | 62.4 |
| According to example 3 | 58.7 | 0 | 41.3 |
| According to example 4 | 90.2 | 0 | 9.8 |
| According to example 5 | 90.0 | 0 | 10.0 |
| According to example 6 | 84.4 | 0.033 | 15.6 |
| According to example 7 | 84.3 | 0.081 | 15.6 |
| According to example 8 | 79.9 | 0.072 | 20.0 |
| According to example 9 | 82.3 | 0.166 | 17.5 |
| According to example 10 | 29.0 | 0.026 | 70.9 |
| According to example 11 | 27.4 | 0.050 | 72.6 |
| According to example 12 | 31.2 | 0.053 | 68.7 |

TABLE 2
Ratio of components in the resulting combinations

| | Composition of a combination | | | |
|---|---|---|---|---|
| | Component | | Polymer content, | Salt content, |
| Sample | Name | wt. % | wt. % | wt. % |
| According to | OSP | 10⁻⁴ | 85.0 | 15.0 |
| example 13 | | | | |
| According to | Echinacea | 2.7 | 77.3 | 20.0 |
| example 14 | extract | | | |
| According to | Licorice | 3.2 | 77.0 | 19.8 |
| example 15 | extract | | | |
| According to | Ginger | 3.1 | 77.6 | 19.3 |
| example 16 | extract | | | |
| According to | Elecampane | 2.6 | 78.0 | 19.4 |
| example 17 | extract | | | |

Example of a Pharmaceutical Preparation

Tablets were produced by a traditional method, namely by mixing ingredients and tableting on a tableting machine by direct compression. Qualitative and quantitative compositions of the tablets are given in Table 3.

TABLE 3

| Components | | | | | |
|---|---|---|---|---|---|
| | | Potato | Calcium | | |
| Active agent | | starch | stearate | Ludipress* | Tablet |
| | Amount, | Amount, | Amount, | Amount, | mass |
| Type | g (%) | g (%) | g (%) | g (%) | (g) |
| Compound | 0.005 | 0.02 | 0.0008 | 0.0742 | 0.1 |
| according to | (5%) | (20%) | (0.8%) | (74.2%) | |
| example 8 | | | | | |
| Compound | 0.01 | 0.02 | 0.0008 | 0.0692 | 0.1 |
| according to | (10%) | (20%) | (0.8%) | (69.2%) | |
| example 8 | | | | | |
| Compound | 0.02 | 0.02 | 0.0008 | 0.0592 | 0.1 |
| according to | (20%) | (20%) | (0.8%) | (59.2%) | |
| example 8 | | | | | |
| Compound | 0.015 | 0.03 | 0.0012 | 0.1038 | 0.15 |
| according to | (10%) | (20%) | (0.8%) | (69.2%) | |
| example 8 | | | | | |
| Compound | 0.02 | 0.04 | 0.0016 | 0.1384 | 0.2 |
| according to | (10%) | (20%) | (0.8%) | (69.2%) | |
| example 8 | | | | | |
| Compound | 0.04 | 0.04 | 0.0016 | 0.1184 | 0.2 |
| according to | (20%) | (20%) | (0.8%) | (59.2%) | |
| example 8 | | | | | |
| Compound | 0.005 | 0.02 | 0.0008 | 0.0742 | 0.1 |
| according to | (5%) | (20%) | (0.8%) | (74.2%) | |
| example 1 | | | | | |
| Compound | 0.01 | 0.02 | 0.0008 | 0.0692 | 0.1 |
| according to | (10%) | (20%) | (0.8%) | (69.2%) | |
| example 1 | | | | | |
| Compound | 0.02 | 0.02 | 0.0008 | 0.0592 | 0.1 |
| according to | (20%) | (20%) | (0.8%) | (59.2%) | |
| example 1 | | | | | |
| Compound | 0.015 | 0.03 | 0.0012 | 0.1038 | 0.15 |
| according to | (10%) | (20%) | (0.8%) | (69.2%) | |
| example 1 | | | | | |
| Compound | 0.02 | 0.04 | 0.0016 | 0.1384 | 0.2 |
| according to | (10%) | (20%) | (0.8%) | (69.2%) | |
| example 1 | | | | | |
| Compound | 0.04 | 0.04 | 0.0016 | 0.1184 | 0.2 |
| according to | (20%) | (20%) | (0.8%) | (59.2%) | |
| example 1 | | | | | |
| Compound | 0.005 | 0.02 | 0.0008 | 0.0742 | 0.1 |
| according to | (5%) | (20%) | (0.8%) | (74.2%) | |
| example 13 | | | | | |
| Compound | 0.01 | 0.02 | 0.0008 | 0.0692 | 0.1 |
| according to | (10%) | (20%) | (0.8%) | (69.2%) | |
| example 13 | | | | | |
| Compound | 0.02 | 0.02 | 0.0008 | 0.0592 | 0.1 |
| according to | (20%) | (20%) | (0.8%) | (59.2%) | |
| example 13 | | | | | |
| Compound | 0.015 | 0.03 | 0.0012 | 0.1038 | 0.15 |
| according to | (10%) | (20%) | (0.8%) | (69.2%) | |
| example 13 | | | | | |
| Compound | 0.02 | 0.04 | 0.0016 | 0.1384 | 0.2 |
| according to | (10%) | (20%) | (0.8%) | (69.2%) | |
| example 13 | | | | | |
| Compound | 0.04 | 0.04 | 0.0016 | 0.1184 | 0.2 |
| according to | (20%) | (20%) | (0.8%) | (59.2%) | |
| example 13 | | | | | |
| Compound | 0.005 | 0.02 | 0.0008 | 0.0742 | 0.1 |
| according to | (5%) | (20%) | (0.8%) | (74.2%) | |
| example 9 | | | | | |
| Compound | 0.01 | 0.02 | 0.0008 | 0.0692 | 0.1 |
| according to | (10%) | (20%) | (0.8%) | (69.2%) | |
| example 9 | | | | | |
| Compound | 0.02 | 0.02 | 0.0008 | 0.0592 | 0.1 |
| according to | (20%) | (20%) | (0.8%) | (59.2%) | |
| example 9 | | | | | |
| Compound | 0.015 | 0.03 | 0.0012 | 0.1038 | 0.15 |
| according to | (10%) | (20%) | (0.8%) | (69.2%) | |
| example 9 | | | | | |
| Compound | 0.02 | 0.04 | 0.0016 | 0.1384 | 0.2 |
| according to | (10%) | (20%) | (0.8%) | (69.2%) | |
| example 9 | | | | | |
| Compound | 0.04 | 0.04 | 0.0016 | 0.1184 | 0.2 |
| according to | (20%) | (20%) | (0.8%) | (59.2%) | |
| example 9 | | | | | |

*Ludipress is a direct compression lactose, composition: lactose monohydrate, povidone (Kollidon 30), crospovidone (Kollidon CL)

When testing the solubility, it was found that in dissolving a tablet in 500 ml of water by stirring using a mixer at 100 rpm, 100% of the active substance passes into the solution in 45 minutes.

The developed methods for preparing compositions, due to the additional steps with well-defined control points, provide products with improved physicochemical and biological characteristics relative to the starting polyphenols and, despite for a limited number of the starting compounds, allow a significant expansion of the structural diversity of products.

The examples given in Table 4 show that, depending on the conditions, the use of the same starting components provides compositions with different qualitative and quantitative content of the resulting polyphenols (example 8 and 9) and polymer component (example 4 and 5).

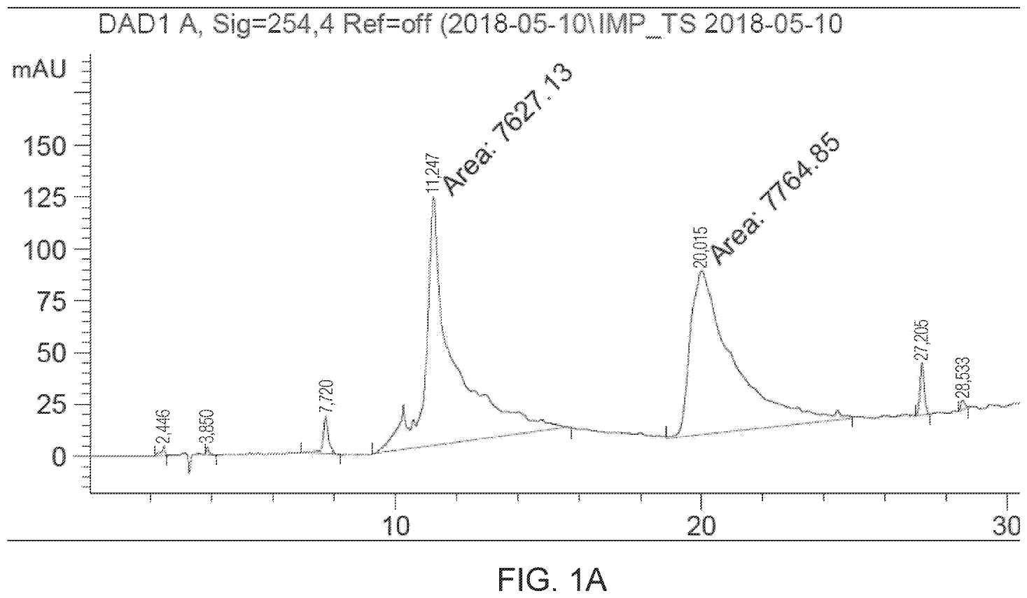

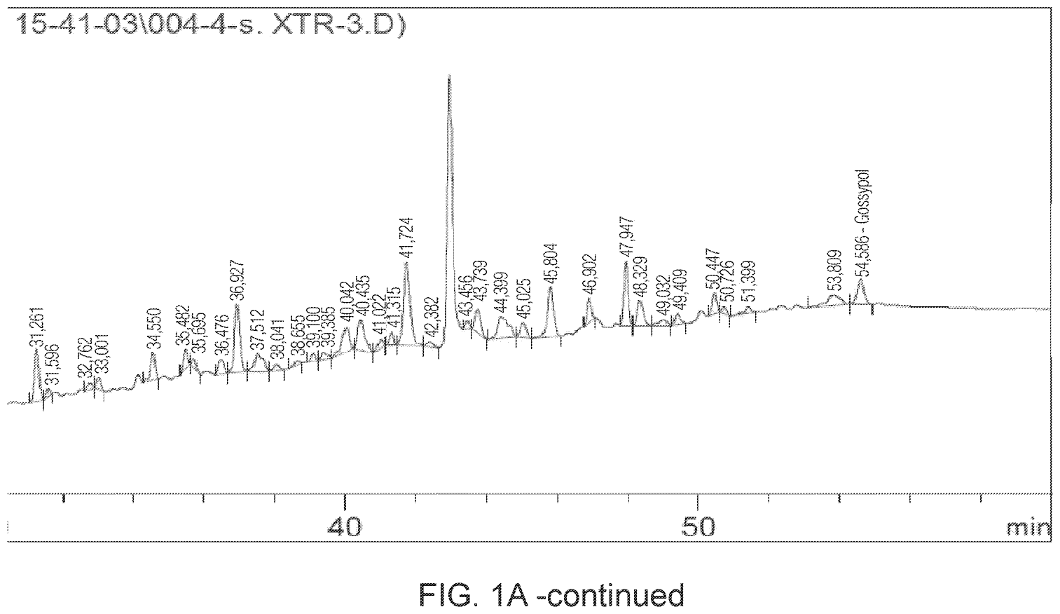

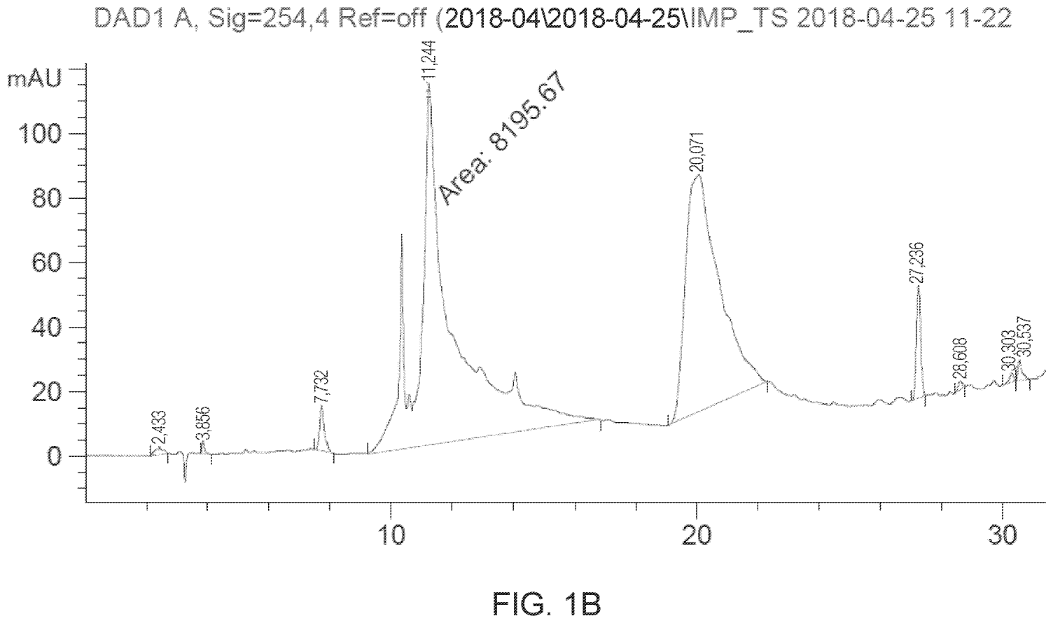

In particular, when the process runs under strongly alkaline conditions, polyphenols that are unstable under alkaline conditions are transformed into their structural analogs. Chromatograms of compositions with modifications of the introduced polyphenol are presented in FIG. 1.

TABLE 4
Characteristics of compositions by the content of polyphenols

| | | | | | Molecular |
|---|---|---|---|---|---|
| | Starting | Carbonyl | | | weight range |
| | polymer | group | Polyphenol | Content of | of polyphenol |
| | and | content, | content, | polyphenol | components, |
| Sample | polyphenol | mmol/g | wt. | components | Da |
| Example 8 | DACMC/ | 0.84 | 0.072 | 20 | 349-541 |
| | Gossypol | | | | |
| Example 9 | DACMC/ | 0.88 | 0.166 | 30 | 325-517 |
| | Gossypol | | | | |
| Example 4 | DAD/ | 0.75 | 0 | 0 | — |
| | Gossypol | | | | |
| Example 5 | DAD/ | 0.54 | 0 | 0 | — |
| | Gossypol | | | | |

An additional advantage of the developed methods is the possibility of introducing auxiliary substances (for example, sodium carbonate) into the compositions during the synthesis, rather than by subsequent addition to the finished pharmaceutical substance. In addition, the above methods allow the biological properties to be adjusted by preparing combinations with both other pharmaceutical substances (example 13) and extracts of medicinal plants (examples 14-17).

The structure and quantitative content of the resulting compositions were studied by methods of HPLC-UV-MS, NMR, GPC, IR spectrometry, and potentiometric titration.

The aromatic components introduced into the polymer were identified according to the standard of the corresponding polyphenols or by the retention time, mass- and UV spectra. The content of unidentified polyphenols in the resulting compositions was determined by HPLC, making calculations according to gossypol serving as an external standard due to the lack of standards for each of the individual substances.

Since HPLC chromatograms of products, which are compositions, differ from the corresponding chromatograms of blank samples (products without introduced polyphenols) only by the presence of peaks of individual polyphenols, it can be said that there is no covalent bonds between the components of the composition.

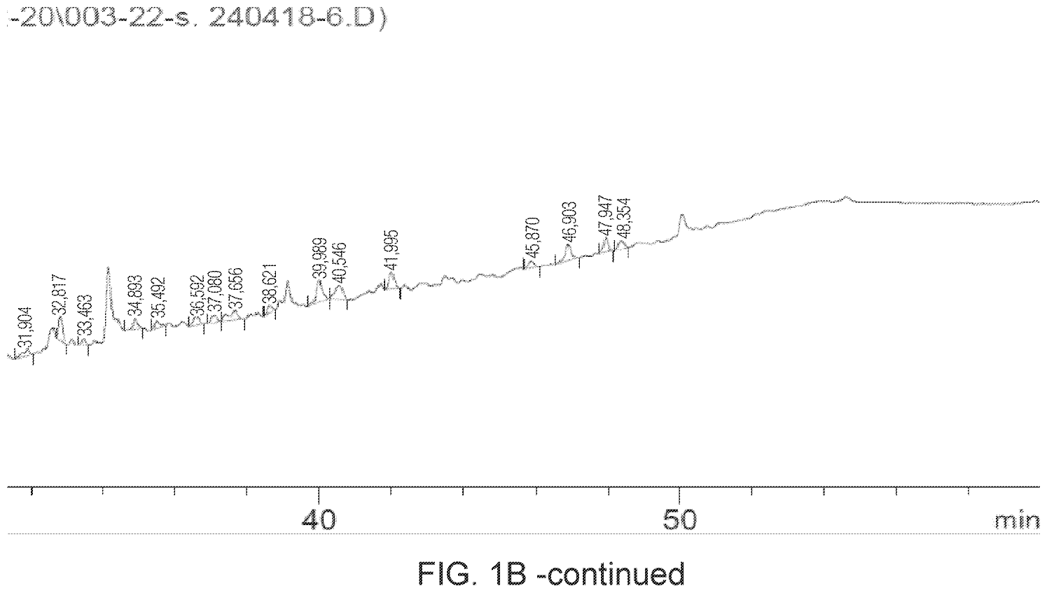

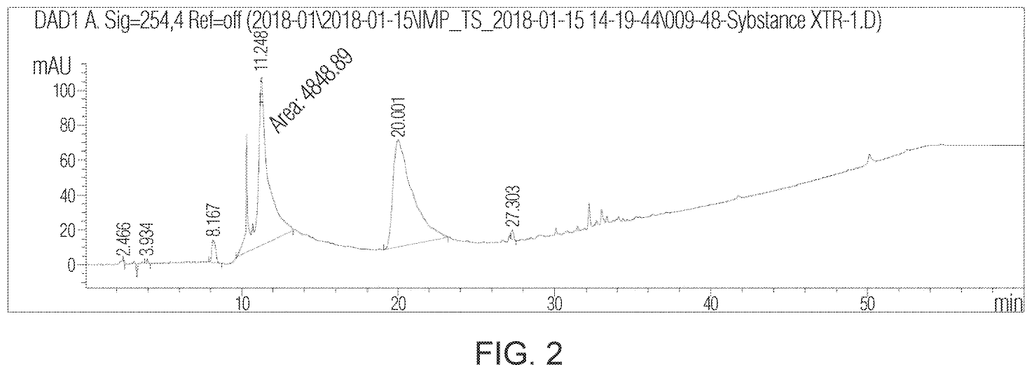

The chromatogram of the composition obtained without the introduction of polyphenols is presented in FIG. 2 (detection was made at 254 nm).

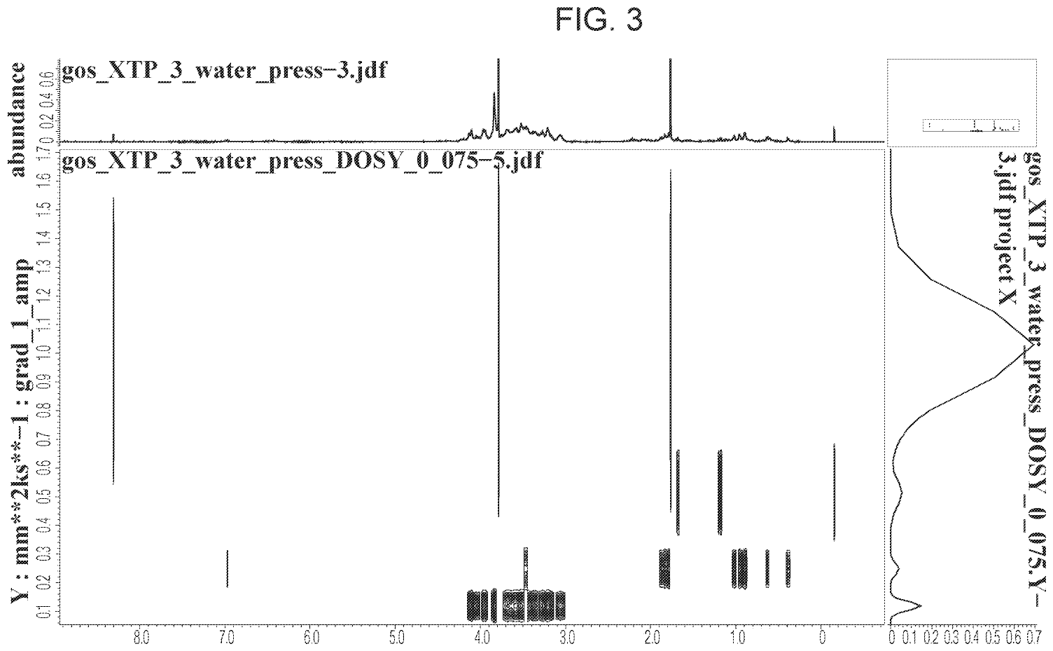

An additional confirmation of the absence of a covalent bond between polymer and polyphenols is the NMR analysis of the composition presented in FIG. 3. For the sample of example 9, a DOSY spectrum was recorded, and this spectrum shows a difference in self-diffusion coefficients between the polymer (group of signals in the range of 3-5 ppm) and polyphenols (aromatic signals in the range of 6 to 11 ppm, and aliphatic signals in the range of 2.5 to 0.5 ppm).

During the preparation of the compositions, the structure of the polymer part undergoes major changes only as related to carbonyl groups. Table 5 shows examples of the compositions for which the conditions were selected in such a way that the polymer part was modified to a various extent, which is reflected in the content of aldehyde groups compared to the starting polymer.

TABLE 5
Characteristics of the compositions
by the content of aldehyde groups

| | Content of | Content of | Content of carbonyl |
|---|---|---|---|
| | aldehyde groups | aldehyde groups | groups compared to |
| | in polymer, | in a composition, | the starting polymer, |
| Sample | mmol/g | mmol/g | % |
| Example 5 | 1.60 | 0.54 | 33.8 |
| Example 9 | 0.94 | 0.87 | 92.6 |

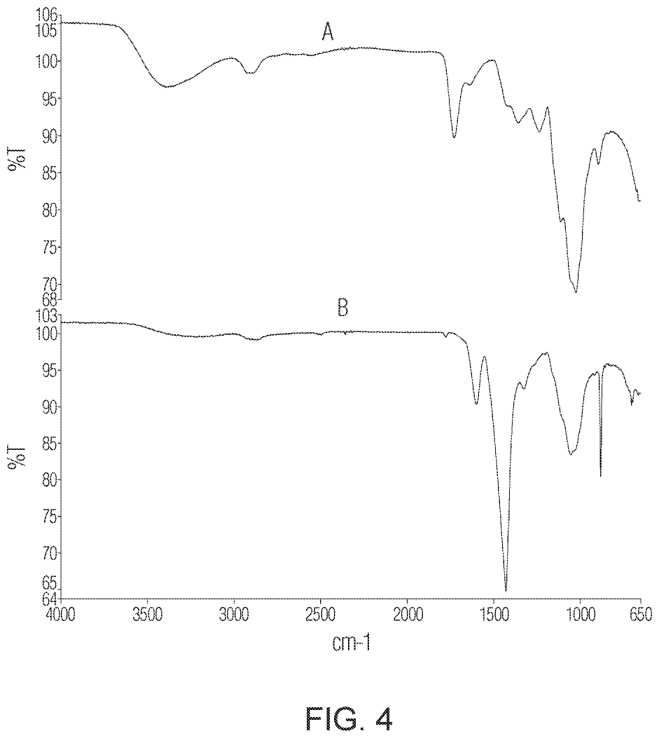

FIG. 4 shows the IR spectrum of the composition (example 8) in comparison with the starting polymer. It can be seen that the form of the IR spectrum of the composition is almost completely determined by the polymer component. Compared to the IR spectrum of the starting DACMC, there are no significant changes in the position and intensity of the absorption bands, except for the characteristic band at 1727 cm⁻¹ corresponding to the vibrations of carbonyl groups.

The change in the content of aldehyde groups is also reflected in the IR spectrum in which the band at 1727 cm⁻¹ corresponding to the vibrations of the carbonyl groups completely disappeared.

The weight-average molecular weight of the resulting compositions is in the range of 3530 to 39100 Da and is determined by the type of the introduced polymer and the process conditions (Table 6).

TABLE 6
Molecular weight characteristics of the compositions

| | Weight-average |
|---|---|
| Sample | molecular weight, Da |
| According to example 1 | 39100 |
| According to example 2 | 5090 |
| According to example 3 | 3870 |
| According to example 5 | 3530 |
| According to example 6 | 28380 |
| According to example 7 | 29360 |
| According to example 8 | 37320 |
| According to example 10 | 25620 |
| According to example 11 | 25310 |
| According to example 12 | 23790 |
| According to example 14 | 36850 |
| According to example 15 | 36920 |
| According to example 16 | 36890 |
| According to example 17 | 37620 |

The amount of the salt component introduced into the synthesis was determined by potentiometric titration. The amount of the salt in the compositions is fully correlated with the introduced one.

Determination of the Cytotoxic Effect of Substances in MDCK Cell Culture

In the experimental work, MDCK ECACC cell line (Sigma, cat. No. 85011435) at 67-70 passages was used. The cell line was grown in Eagle's MEM (PanEco) containing 10% fetal serum (HyClone), 300 μg/ml L-glutamine, and 0.1 mg/ml normocin.

The MDCK ECACC cells were added to 96-well plates in Eagle's MEM (PanEco) containing 10% fetal serum (HyClone), 300 μg/ml L-glutamine, and 0.1 mg/ml normocin at the rate of 18000 cells/well, cultivated for 24 h, and washed with serum-free medium once before introducing of substances.

For dilution of the test substances, a supporting medium of the following composition was used: Eagle's MEM (PanEco) containing 2% fetal serum (HyClone), 300 μg/ml L-glutamine, 12 μg/ml chymopsin-trypsin, and 0.1 mg/ml normocin.

Each experimental condition was tested in 4 parallel wells. The last dilution was placed in the first 2 of 4 wells, and the supporting medium was added to the last two of 4 wells (cell control).

The cells were incubated with test substances for 48 hours in a CO₂ incubator at 37° C., after which the culture medium was removed, and 100 μl of the supporting medium and 20 μl of MTS solution (Promega, cat. No. G3581) were added to each well. After incubation for 3 hours at 37° C., the optical density was determined at a wavelength of 492 nm and a reference wavelength of 620 nm, using a BIO-RAD microplate spectrophotometer. The concentration of a test substance, which reduces the value of optical density by 50% compared with the control cells, was taken as a 50% cytotoxic dose (CC₅₀).

Study of the Effect of Substances on Infectious Titer of Influenza Virus in MDCK Cell Culture

Influenza A/Puerto-Rico/8/34 (H1N1) virus adapted to the MDCK line was used in the studies. The infectious and hemagglutination activity of the virus were determined by the methods recommended by WHO.

The virus-specific effect of the test substances was studied against A/Puerto-Rico/8/34 (H1N1) strain of influenza virus in MDCK ECACC cells, using Eagle's MEM (PanEco) containing 2% fetal serum (HyClone), 300 μg/ml L-glutamine, 12 μg/ml of chymopsin-trypsin, and 0.1 mg/ml of normocin. The MDCK ECACC cell culture was prepared in the same way as in the experiments on determination of the cytotoxic effect of studied substances. Before infection with the virus, MDCK cells were washed once with serum-free Eagle's MEM, then 100 μl of the preliminarily prepared in the supporting medium dilutions of substances (single concentration) were added into the wells and incubated for 1 h at 37° C. After that, 10 μl of preliminarily prepared 10-fold dilutions of the virus were added. Cell and viral controls were done similarly, using the same medium. The results were assessed after 48 h according to CPE and hemagglutination reaction (HAR). In the HAR, 0.75% suspension of human erythrocytes (blood group 0) in saline was used.

50% of the cytotoxic concentration (CC₅₀) and 50% of the inhibiting concentration (IC₅₀) for each of the studied compounds were calculated using Excel and GraphPad Prism 6.01 software package. A 4-parameter logistic curve equation was taken as the working model for analysis of the CC₅₀ (menu items “Nonlinear regression”-“Sigmoidal dose-response (variable slope)”). For analysis of the IC₅₀ a similar 4-parameter logistic curve equation (the menu items “Nonlinear regression”-“log (inhibitor) vs. response (variable slope)”) was taken. Virus inhibitory effect of the studied substances (ΔlgTCID₅₀) was assessed on decline of viral infection titer in the experimental wells compare with control wells, and calculated by the Reed and Muench method. The results are given in Table 7.

TABLE 7

| | | Antiviral activity against | |
|---|---|---|---|
| | Cytotoxicity | A/PR8 (H1N1) virus | |
| Sample | CC_{50 av}, mg/ml | IC_{50 av} | ΔlgTCID_{50 av}* |
| Gossypol acetic acid | 0.0039 | 0.0012 | 1.32 |
| Apogossypol | 0.0094 | 0.0000 | 0.50 |
| According to example 1 | 26.3100 | 0.1384 | 3.00 |
| According to example 2 | 9.4960 | 0.0418 | 1.75 |
| According to example 6 | 22.8450 | 0.7944 | 2.38 |
| According to example 8 | 16.1564 | 0.7769 | 1.64 |
| According to example 9 | 2.85 | 1.52 | 1.00 |
| According to example 10 | 8.3150 | 0.9215 | 1.00 |
| According to example 11 | 8.4640 | 0.9480 | 1.00 |
| According to example 12 | 5.7670 | 0.0069 | 1.75 |
| According to example 13 | >60 | 0.3103 | 2.88 |
| According to example 14 | 27.8100 | 4.7350 | 1.00 |
| According to example 15 | 29.6600 | 0.1370 | 2.00 |
| According to example 16 | 29.4900 | 1.4950 | 1.00 |
| According to example 17 | 29.4100 | 0.1403 | 1.50 |

*Substances with pronounced antiviral activity have ΔlgTCID_{50 av.} ≥ 1.50.

In comparison with the known polyphenols (gossypol and apogossypol), all tested samples have significantly lower cytotoxicity values at comparable or higher activity values.

Study of the Antimicrobial Activity of the Substances Against S. aureus

The experiments were carried out according to the Guidelines for conducting of preclinical studies (edited by Mironov, 2012).

The minimal inhibitory (suppressive, bacteriostatic) concentration (MIC) and the minimal bactericidal concentration (MBC) were determined.

1. Staphylococcus aureus (Clinical Isolate)

The study with a clinical staphylococcus strain was performed by using a staphylococcus infective dose of 10⁵ CFU/ml. The substances according to examples 1, 8, and 9 were diluted to 100 mg/ml by using equal volumes of DMSO and water for injection. The start solutions of substances were passed through a syringe filter with PES-membrane. The MIC value was determined by the serial dilution method in 96-well flat-bottom plates by co-cultivation of the substance dilutions and the test strain in a liquid nutrient medium, using two replications. The MIC value was determined as the lowest concentration of the substance, which inhibits visible microorganism growth. To determine the MBC, content of the wells after incubation were sown on agar medium. The results are given in Table 8.

TABLE 8

| | According to | | According to | | According to | |
|---|---|---|---|---|---|---|
| Microorganism | example 1 | | example 8 | | example 9 | |
| concentration, | MIC | MBC | MIC | MBC | MIC | MBC |
| CFU/ml | mg/ml | mg/ml | mg/ml | mg/ml | mg/ml | mg/ml |
| 10⁵ | 25 | >50 | 25 | >50 | 25 | 50 |

For the substances according to examples 1 and 8, in co-cultivation with staphylococcus at a concentration of 10⁵ CFU/ml, MIC value was 25 mg/ml, and MBC value was not determined (it was higher than the concentration of the substances in the initial well). For the substance according to example 9, MIC value was 25 mg/ml, and MBC value was 50 mg/ml.

2. Staphylococcus aureus 6538P (Reference Strain)

The study with a reference staphylococcus strain (Staphylococcus aureus 6538P) was performed using different infectious doses of staphylococcus (10³, 10⁴, 10⁵, 10⁶, and 10⁷ CFU/ml). The substances according to examples 1 and 9 were diluted to 100 mg/ml, and the substance according to example 8 was diluted to 200 mg/ml by using equal volumes of DMSO and water for injection. The diluted preparations were stirred and dissolved in a water bath at 37° C. for 2 hours. The start solutions of substances were passed through a syringe filter with PES-membrane. The substances according to examples 1 and 9 were initially diluted to 200 mg/ml, but since they could not be filtered, the solutions were additionally diluted two times.

The MIC value was determined by the serial dilution method in a 96-well flat-bottom plates by co-cultivation of the substance dilutions and the test strain in a liquid nutrient medium, using two replications. The MIC value was determined as the lowest concentration of the substance, which inhibits visible microorganism growth. To determine the MBC, content of the wells after incubation from the wells were sown on agar medium. The results are given in Table 9.

TABLE 9

| | According to | | According to | | According to | |
|---|---|---|---|---|---|---|
| Microorganism | example 1 | | example 8 | | example 9 | |
| concentration, | MIC | MBC | MIC | MBC | MIC | MBC |
| CFU/ml | mg/ml | mg/ml | mg/ml | mg/ml | mg/ml | mg/ml |
| 10³ | ND* | ND | 25 | 100 | 25 | >50 |
| 10⁴ | 12.5 | >50 | 50 | 100 | 25 | >50 |
| 10⁵ | 25 | >50 | 50 | 100 | 25 | >50 |
| 10⁶ | 25 | >50 | 50 | >100 | 25 | >50 |
| | | | | | (filter) | (filter) |
| | | | | | 6.25 | 50 |
| | | | | | (non-filtered) | (non-filtered) |
| 10⁷ | ND | ND | 50 | >100 | 25 | >50 |
| | | | | | (filtered) | (filtered) |
| | | | | | 12.5 | 50 |
| | | | | | (non-filtered) | (non-filtered) |

*not determined

When comparing the effects of the substance according to example 9 on staphylococcus at its concentrations 10⁶ and 10⁷ CFU/ml, it was found that the MIC and MBC values of the substances are different before and after filtration. Under the impact of substances on staphylococcus at its concentration 10⁶ CFU/ml, the MIC value reduced 4 times after filtration (6.25 mg/ml before filtration, and 25 mg/ml after filtration); MBC value was not determined after filtration (>50), while before filtration it was 50 mg/ml. Under the impact of substances on staphylococcus at its concentration 10⁷ CFU/ml, the MIC value reduced 2 times after filtration (12.5 mg/ml before filtration, and 25 mg/ml after filtration); MBC value was not determined after filtration (>50), while before filtration it was 50 mg/ml.

1-18. (canceled)

19. An antimicrobial composition containing a biologically effective amount of an active component, wherein the active component is a product based on a polysaccharide and a polyphenol, not linked by covalent bonds, wherein the product is obtained by:
pretreatment of the initial polysaccharide selected from the group consisting of dialdehyde carboxymethyl cellulose and dialdehyde dextran, in an aqueous or aqueous-organic medium at a pH of 10 to 14 and at a temperature of 10 to 60° C. to obtain a treated polysaccharide containing carbonyl groups from 95 to 20% based on the initial polysaccharide, optionally with additional purification and/or acidification and/or desalting and/or fractionation steps, and
pretreatment of the initial polyphenol selected from the group consisting of apogossypol, gossypolone, apogossypolone, gossypol, gossypolacetic acid, 1,1′,6,6′,7,7′-hexahydroxy-5,5′-diisopropyl-3,3′-dimethyl-2,2′-binaphthalene-8-carbaldehyde, 6,6′,7,7′-tetrahydroxy-5,5′-diisopropyl-3,3′-dimethyl-1,1′,4,4′-tetraoxo-1,1′,4,4′-tetrahydro-2,2′-binaphthalene-8-carbaldehyde, in an aqueous or aqueous organic medium at a pH in the range from 10 to 14 and at a temperature of 10 to 60° C. to obtain a treated polyphenol having a molecular weight of 300 to 550 atomic mass units, optionally with additional purification and/or acidification and/or desalting and/or fractionation steps,
followed by combining the treated polyphenol and the treated polysaccharide and co-treated them in an aqueous or aqueous-organic medium at a pH of 10 to 14 and at a temperature of 10 to 60° C. and steps of acidification, isolation and purification of the resulting product;
and pharmaceutically acceptable excipients.

20. An antimicrobial composition containing:
a biologically effective amount of an active component, wherein the active component is a product obtained by treatment of the initial polysaccharide selected from the group consisting of dialdehyde carboxymethyl cellulose and dialdehyde dextran, in an aqueous or aqueous-organic medium at a pH of 10 to 14 and at a temperature of 10 to 60° C. to obtain a treated polysaccharide containing carbonyl groups from 95 to 20% based on the initial polysaccharide, optionally with additional purification and or acidification and/or desalting and/or fractionation steps, and
and pharmaceutically acceptable excipients.

21. An antimicrobial combination containing an antimicrobial composition according to claim 19 or 20 and at least one extract of plant materials, selected from the group consisting of elecampane, ginger, licorice roots) and purpurea echinacea, wherein the content of the extract in the combination is from 0.01 to 99.99%;
and pharmaceutically acceptable excipients.

22. Antimicrobial combination according to claim 21, wherein the extract content of the combination is 1.0 to 50.0%, preferably 1.0 to 10.0%.

23. An antimicrobial combination containing a composition according to claim 19 or 20 and Oseltamivir, wherein the content of Oseltamivir in the combination is from 0.0000001 to 99.9999999%.

24. The antimicrobial combination according to claim 23, wherein the oseltamivir content is 0.0000001 to 10.0%, preferably 0.0001 to 5.0%.

25. A method for preparing a composition according to claim 19, comprising:
a step of pretreatment of the initial polysaccharide selected from the group consisting of dialdehyde carboxymethyl cellulose and dialdehyde dextran, in an aqueous or aqueous-organic medium at a pH of 10 to 14 and at a temperature of 10 to 60° C. to obtain a treated polysaccharide containing carbonyl groups from 95 to 20% based on the initial polysaccharide, optionally with additional purification and/or acidification and/or desalting and/or fractionation steps
a step of pretreatment of the initial polyphenol selected from the group consisting of apogossypol, gossypolone, apogossypolone, gossypol, gossypolacetic acid, 1,1′,6,6′,7,7′-hexahydroxy-5,5′-diisopropyl-3,3′-dimethyl-2,2′-binaphthalene-8-carbaldehyde, 6,6′,7,7′-tetrahydroxy-5,5′-diisopropyl-3,3′-dimethyl-1,1′,4,4′-tetraoxo-1,1′,4,4′-tetrahydro-2,2′-binaphthalene-8-carbaldehyde, in an aqueous or aqueous organic medium at a pH in the range from 10 to 14 and at a temperature of 10 to 60° C. to obtain a treated polyphenol having a molecular weight of 300 to 550 atomic mass units, optionally with additional purification and/or acidification and/or desalting and/or fractionation steps,
a step of combining the treated polyphenol and the treated polysaccharide and co-treating them in an aqueous or aqueous-organic medium at a pH of 10 to 14 and at a temperature of 10 to 60° C.,
a subsequent steps of acidification, isolation and purification of a resulting product; and
a final step of combining the resulting product with pharmaceutically acceptable excipients;

26. A method for preparing a treated polysaccharide containing carbonyl groups from 95 to 20% based on the initial polysaccharide, comprising:
a step of pretreatment of the initial polysaccharide selected from the group consisting of dialdehyde carboxymethyl cellulose and dialdehyde dextran, in an aqueous or aqueous-organic medium at a pH of 10 to 14 and at a temperature of 10 to 60° C. to obtain a treated polysaccharide containing carbonyl groups from 95 to 20% based on the initial polysaccharide, optionally with additional purification and/or acidification and/or desalting and/or fractionation steps, and
subsequent stages of acidification, isolation and purification of the resulting product.

27. The method according to claim 25 or 26, wherein the organic solvent is acetone, ethyl alcohol, isopropyl alcohol, 1,4-dioxane, tetrahydrofuran; preferably acetone, ethyl alcohol, isopropyl alcohol.

28. An antimicrobial agent containing a composition according to claim 19 or 20.

29. The antimicrobial agent according to claim 28, wherein the antimicrobial agent is effective against influenza, herpes, hepatitis, and HIV viruses, respiratory viral infections and bacterial infections.

30. Antimicrobial agent containing the combination according to claim 21.

31. Antimicrobial agent according to claim 30, wherein the antimicrobial agent is effective against influenza, herpes, hepatitis, HIV, respiratory viral infections and bacterial infections.
